(19) United States
(12) Patent Application Publication
(10) Pub. No.: US 2015/0164680 A1
(43) Pub. Date: Jun. 18, 2015

(54) SANITARY PRODUCT STRUCTURE FOR HUMAN BODY WITH FUNCTIONS OF STERILIZATION AND DEODORIZATION

(71) Applicant: TENG-LAI CHEN, KAOHSIUNG CITY (TW)

(72) Inventor: TENG-LAI CHEN, KAOHSIUNG CITY (TW)

(21) Appl. No.: 14/105,320

(22) Filed: Dec. 13, 2013

(51) Int. Cl. A61F 5/455; A61F 13/20; A61F 13/53

(52) U.S. Cl. CPC A61F 5/4553; A61F 13/53; A61F 13/2051; A61F 2013/51076

(57) ABSTRACT

The invention relates to a sanitary product structure for human body with functions of sterilization and deodorization. It comprises a main body and at least one metal ion layer with the functions of sterilization and deodorization. The metal ion layer is at least disposed on the surface of the main body, wherein the metal ion layer is a cover layer composed of germanium ions, molybdenum ions, magnesium ions, manganese ions, chromium ions, vanadium ions, zinc ions, silicon ions, platinum ions, aluminum ions, selenium ions, calcium ions, titanium ions or any combinations thereof. Accordingly, the human body sanitary product has the effects of sterilization and deodorization.

### BACKGROUND OF THE INVENTION

1. Fields of the Invention

The present invention relates to a sanitary product structure for human body with functions of sterilization and deodorization, especially to a kind of skin-friendly sanitary product showing the effects of sterilization and deodorization.

2. Descriptions of Related Art

In general, skin-friendly sanitary products for use in human bodies include tampons, sanitary pads, diapers, panty-shaped diapers, incontinence pads, toilet papers, wet towels, bath towels, shower sponges, towels, menstrual cups and so on, wherein tampons are mainly used to absorb the metabolized menses in female menstrual cycle and sanitary pads used to absorb female vaginal discharge. Therefore, tampons and sanitary pads are imperative for modern women; likewise, diaper, panty-shaped diapers and incontinence pads are either worn by children who are not yet potty trained or experience bedwetting, or used by adults with incontinence or in certain circumstances where access to a toilet is unavailable for purposes of absorbing urine or receiving excrement. These adults can include the elderly or those with a physical or mental disability.

According to the survey, most of women suffered from various gynecological inflammations. Mainly due to the hormones change in menstrual cycles, the acidity of a vagina will get weakened. Additionally, in case of a tampon with a bad air permeability used by female for a long time, the female user's private part will stay under a high-temperature environment and make the tampon become a best hotbed for culturing bacteria to cause discomfort to the female user, such as prevailing gynecological diseases resulted from symptoms of severe odor, genital itching or abnormal secretion. These symptoms will get more and more serious in the hot and wet weather and also seriously affected the normal; life and works of women.

The same situations also occurred in other skin-friendly sanitary products, such as the diaper, panty-shaped diaper and incontinence pad. The issues of hot, humid, severe odor, bacterial infection and itching also happened after using these sanitary products, wherein as far as the diaper, because that the wear time is longer, there are several times of excretion during wear and the humidity inside the diaper becomes high after mixing the sweat, a diaper rash is easily produced due to hot. If one does not change the diaper frequently, he may be complicated by candidiasis. Candida is one kind of the fungus, it likes to grow in humid environment, so the wet diaper and skin temperature are the hotbed of candida breeding.

In addition, because that the toilet paper, wet towel, bath towel, shower sponge and towel used to wipe the face and body are placed or hung in a poorly ventilated toilet or bathroom, the bacteria easily proliferate. When these skin-friendly products of the toilet paper, wet towel, bath towel, shower sponge and towel are used to wipe face and body, the body is easily infected by the bacteria.

Furthermore, there is a kind of the menstrual cup to collect the menses in the market. The menstrual cup has an overall shape similar to a small goblet having a short handle at a bottom thereof. The menstrual cup is soft and flexible, so that it is foldably put into the vagina for collecting the menses. The menstrual cup has different capacity, it can be removed and rinsed after placing four to eight hours, and it will be repeated use for five to ten years if it is properly maintained. Because that the woman can clearly understand the amount of the menses through the menstrual cup, and the menstrual cup uses the way of the received instead of absorption to collect the menses, comparing to the tampons, the menstrual cup does not cause the situations of damage or dry on the vaginal mucosa. The menstrual cup does not cause any allergy as the one induced by the bleach and deodorant in the tampon. The menses does not contact with air, so there is no any problem of the odor. The menstrual cup can be reused after cleaned, so it has the advantageous features of the environmental protection and saving money. Therefore, the menstrual cup made of medical silicone or natural rubber becomes a new choice for female under menstrual cycles.

Although the menstrual cup has above advantages, female consumers still have many questions in using thereof, for example, the menstrual cup washed with water only and repeated placed for many times into the vagina, which would cause any possible infection. Thus, it is a significant low possibility for female consumers to accept using the menstrual cup.

### SUMMARY OF THE INVENTION

Therefore, a sanitary product structure is developed herein to enhance the sterilized ability of skin-friendly sanitary products, such as tampons, sanitary pads, diapers, panty-shaped diapers, incontinence pads, toilet papers, wet towels, bath towels, shower sponges, towels and menstrual cups. A primary goal of the present invention is to provide a sanitary product structure for human body with functions of sterilization and deodorization. The safety and hygiene for use of sanitary products on a human body can be enhanced due to the sterilizing and deodorizing effects from the sanitary products.

In order to achieve the above objectives, a sanitary product structure for human body with functions of sterilization and deodorization of the present invention is revealed herein. It includes a main body and at least one metal ion layer with the functions of sterilization and deodorization disposing on at least one surface of the main body, the at least one metal ion layer is a cover layer composed of germanium ions, molybdenum ions, magnesium ions, manganese ions, chromium ions, vanadium ions, zinc ions, silicon ions, platinum ions, aluminum ions, selenium ions, calcium ions, titanium ions or any combination thereof. With the at least one metal ion layer disposed in the sanitary product, the sanitary product enables to show the effects of sterilization and deodorization for further enhancement of safety and hygiene in use thereof.

At least one surface of the main body in the present invention is preferably refers to a surface where a human body will contact.

The sanitary product in the present invention includes tampons, sanitary pads, diapers, panty-shaped diapers, incontinence pads, toilet papers, wet towels, bath towels, shower sponges, towels and menstrual cups.

### BRIEF DESCRIPTION OF THE DRAWINGS

The structure and the technical means adopted by the present invention to achieve the above and other objects can be best understood by refereeing to the following detailed description of the preferred embodiments and the accompanying drawings, wherein

FIG. 1 is a partial enlarged drawing showing an embodiment of a human body sanitary product structure according to the present invention;

FIG. 2 is an illustration showing an embodiment of a diaper having a sterilizing and deodorizing metal ion layer according to the present invention;

FIG. 3 is a schematic view showing an embodiment of a panty-shaped diaper having a sterilizing and deodorizing metal ion layer according to the present invention;

FIG. 4 is a schematic view showing an embodiment of a sanitary pad having a sterilizing and deodorizing metal ion layer according to the present invention;

FIG. 5 is a schematic view showing an embodiment of a bath towel having a sterilizing and deodorizing metal ion layer according to the present invention;

FIG. 6 is a schematic view showing an embodiment of a menstrual cup having a sterilizing and deodorizing metal ion layer according to the present invention;

FIG. 7 is a schematic view showing an embodiment of a shower sponge having a sterilizing and deodorizing metal ion layer according to the present invention;

FIG. 8 is a schematic view showing an embodiment of a towel having a sterilizing and deodorizing metal ion layer according to the present invention.

### DETAILED DESCRIPTION OF THE PREFERRED EMBODIMENT

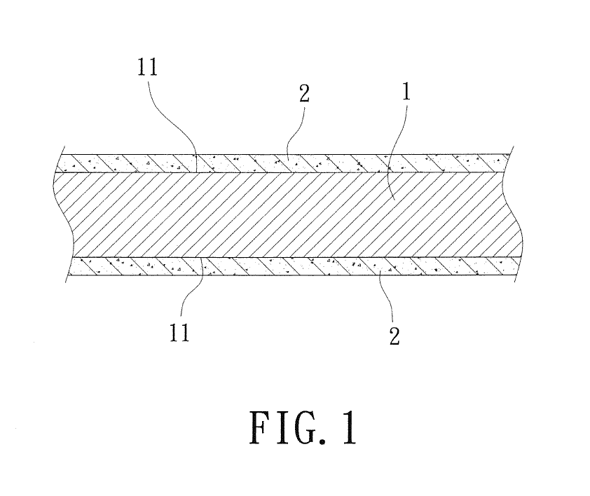

First, please refer to FIG. 1, a partial enlarged drawing showing an embodiment of a human body sanitary product structure according to the present invention, a sanitary product structure with functions of sterilization and deodorization for use on a human body includes a main body (1) and at least one metal ion layer (2) with functions of sterilization and deodorization disposed on at least one surface (11) of the main body (1), wherein the surface (11) refers to a surface with which a human body can get in touch. The at least one metal ion layer (2) is a cover layer composed of germanium ions, molybdenum ions, magnesium ions, manganese ions, chromium ions, vanadium ions, zinc ions, silicon ions, platinum ions, aluminum ions, selenium ions, calcium ions, titanium ions or any combinations thereof.

The sanitary product structure for human body with functions of sterilization and deodorization includes but not limited to the tampons, sanitary pads, diapers, panty-shaped diapers, incontinence pads, toilet papers, wet towels, bath towels, shower sponges, towels and menstrual cups.

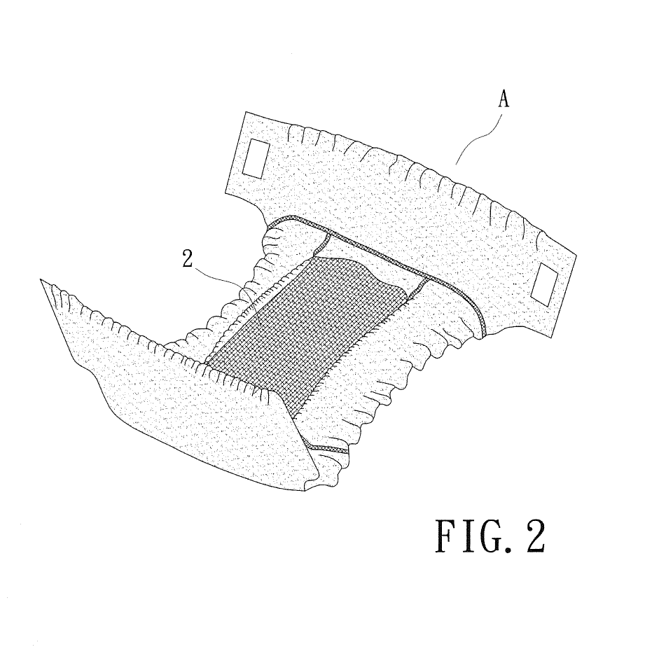

As referring to FIG. 2, it shows a structure of a diaper (A) according to the present invention, wherein the at least one metal ion layer (2) is preferably covered on the entire surface of the diaper (A) with which a human body will get in touch for the effects of sterilization and deodorization.

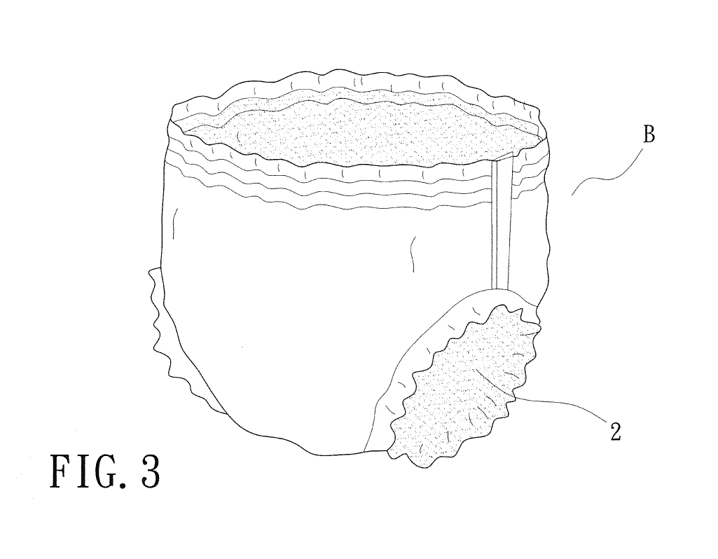

As referring to FIG. 3, it shows a structure of a panty-shaped diaper (B) according to the present invention, wherein the at least one metal ion layer (2) is preferably covered on the entire surface of panty-shaped diaper (B) with which a human body will get in touch for the effects of sterilization and deodorization.

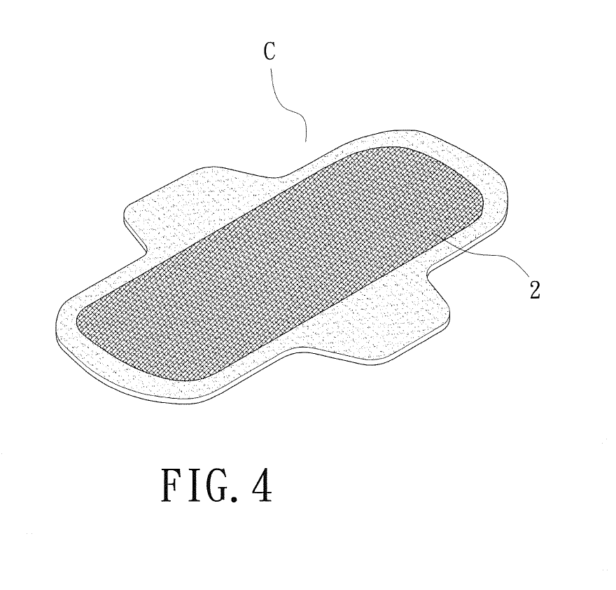

As referring to FIG. 4, it shows a structure of a sanitary pad (C) according to the present invention, wherein the at least one metal ion layer (2) is preferably covered on the entire surface of the sanitary pad (C) with which a human body will get in touch for the effects of sterilization and deodorization.

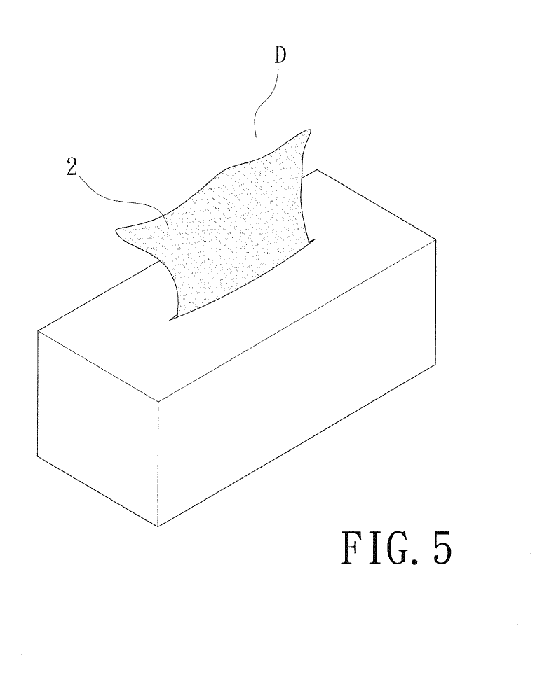

As referring to FIG. 5, it shows a structure of a toilet paper (D) according to the present invention, wherein the at least one metal ion layer (2) is preferably covered on the entire surface of the toilet paper (D) with which a human body will get in touch for the effects of sterilization and deodorization.

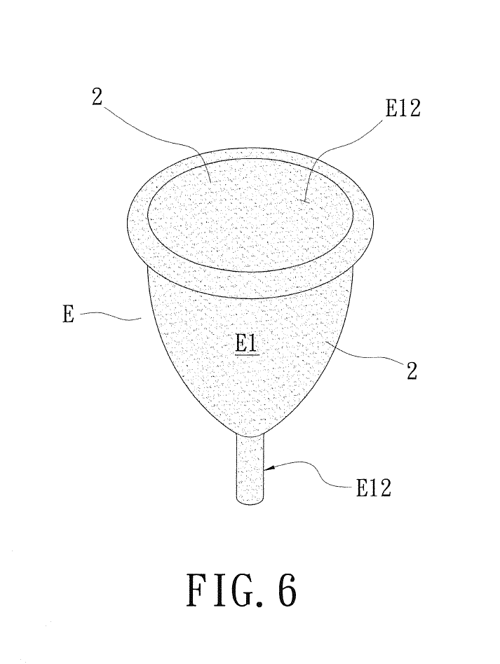

As referring to FIG. 6, it shows a structure of a menstrual cup (E) according to the present invention, wherein the menstrual cup (E) includes a main body (El) made of rubber or silicone in a goblet-like shape. The main body (El) further has an accommodation space (Ell) at an upper portion thereof and a short handle (E12) at a lower portion thereof. The least one metal ion layer (2) with the functions of sterilization and deodorization can be disposed at an outer surface thereof, an inner surface thereof, or both the outer and inner surfaces thereof. The at least one metal ion layer (2) is a cover layer composed of germanium ions, molybdenum ions, magnesium ions, manganese ions, chromium ions, vanadium ions, zinc ions, silicon ions, platinum ions, aluminum ions, selenium ions, calcium ions, titanium ions or any combinations thereof.

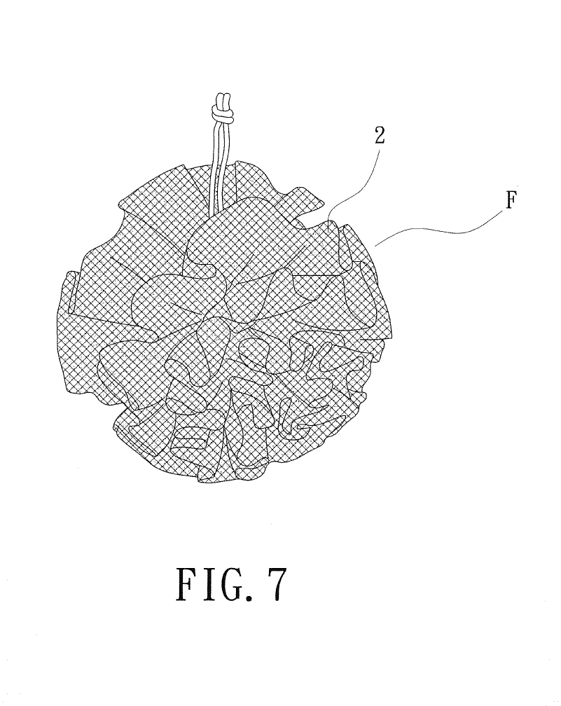

As referring to FIG. 7, it shows a structure of a shower sponge (F) according to the present invention, wherein the at least one metal ion layer (2) is preferably covered on the entire surface of the shower sponge

(F) with which a human body will get in touch for the effects of sterilization and deodorization.

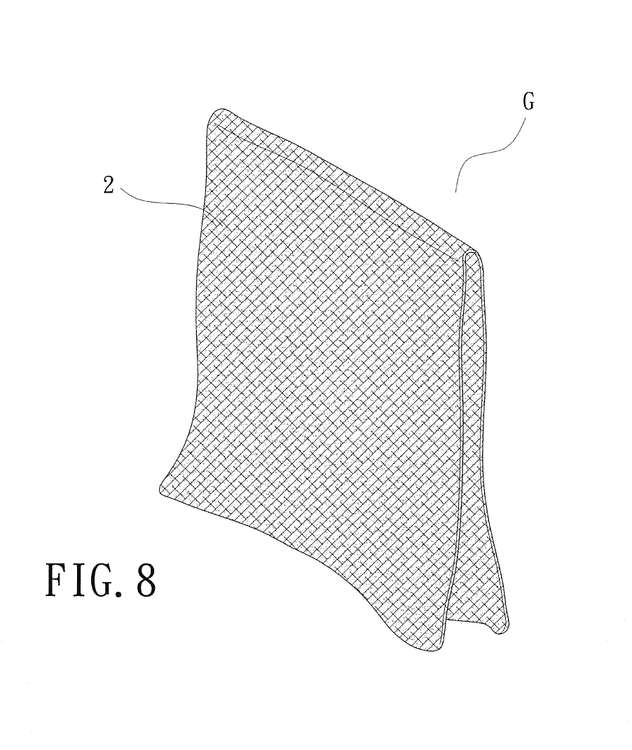

As further referring to FIG. 8, it shows a structure of a towel (G) according to the present invention, wherein the at least one metal ion layer (2) is preferably covered on the entire surface of the towel (G) with which a human body will get in touch for the effects of sterilization and deodorization.

The sanitary product structure of the present invention has the effects of sterilization and deodorization by covering the at least one metal ion layer (2) composed of germanium ions, molybdenum ions, magnesium ions, manganese ions, chromium ions, vanadium ions, zinc ions, silicon ions, platinum ions, aluminum ions, selenium ions, calcium ions, titanium ions or any combinations thereof on the surface of the main body (1) to enhance the safety and hygiene for use of the sanitary products on a human body.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A sanitary product structure for a human body with functions of sterilization and deodorization, comprising:
a main body; and
at least one metal ion layer with functions of sterilization and deodorization disposed on a surface of the main body, wherein the at least one metal ion layer is a cover layer composed of germanium ions, molybdenum ions, magnesium ions, manganese ions, chromium ions, vanadium ions, zinc ions, silicon ions, platinum ions, aluminum ions, selenium ions, calcium ions, titanium ions or any combinations thereof.

2. The structure as claimed in claim 1, wherein the surface of the main body is a surface with which the human body contacts.

3. The structure as claimed in claim 2, wherein the sanitary product includes tampons, sanitary pads, diapers, panty-shaped diapers, incontinence pads, toilet papers, wet towels, bath towels, shower sponges, towels and menstrual cups.

4. The structure as claimed in claim 3, wherein the menstrual cup comprises a main body in a goblet-like shape has an accommodation space at an upper portion thereof and a short handle at a lower portion thereof.

5. The structure as claimed in claim 4, wherein the main body is made of rubber or silicone.

6. The structure as claimed in claim 1, wherein the sanitary product includes tampons, sanitary pads, diapers, panty-shaped diapers, incontinence pads, toilet papers, wet towels, bath towels, shower sponges, towels and menstrual cups.

7. The structure as claimed in claim 6, wherein the menstrual cup comprises a main body in a goblet-like shape has an accommodation space at an upper portion thereof and a short handle at a lower portion thereof.

8. The structure as claimed in claim 7, wherein the main body is made of rubber or silicone.
